(19) United States
(12) Patent Application Publication
(10) Pub. No.: US 2006/0002877 A1
(43) Pub. Date: Jan. 5, 2006

(54) Compositions and methods for permanently reshaping hair using elastomeric film-forming polymers

(76) Inventors: Isabelle Rollat-Corvol, Paris (FR); Jonathan Gawtrey, Boulogne (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/171,223

(22) Filed: Jul. 1, 2005

Related U.S. Application Data
(60) Provisional application No. 60/620,422, filed on Oct. 21, 2004.

(30) Foreign Application Priority Data
Jul. 1, 2004 (FR) 0451399

(51) Int. Cl. A61K 8/87; A61K 8/81; A61K 8/46

(52) U.S. Cl. 424/70.5; 424/70.16; 424/70.17

(57) ABSTRACT

The present disclosure relates to compositions and methods for permanently shaping or for relaxing keratin fibers comprising applying to the keratin fibers a cosmetic composition comprising, in a cosmetically acceptable medium, at least one elastomeric film-forming polymer, wherein the film obtained by drying said at least one film-forming polymer at ambient temperature and at a relative humidity of 55%±5%, has a mechanical profile comprising: (a) an elongation at break (6b) of greater than or equal to 800%,
(b) an instantaneous recovery (Rᵢ) at least equal to 75%, after an elongation of 150%, and
(c) a recovery (R₃₀₀) at 300 seconds of greater than 80%; and at least one compound chosen from keratin-reducing compounds and keratin-fixing compounds.

This application claims the benefit of U.S. Provisional Application No. 60/620,422, filed Oct. 21, 2004, the contents of which are incorporated herein by reference. This application also claims the benefit of priority under 35 U.S.C. § 119 to French Patent Application No. 04 51399, filed Jul. 1, 2004, the contents of which are also incorporated by reference.

The present disclosure relates to compositions and methods for permanently reshaping keratin fibers such as hair. For example, the present disclosure provides compositions and methods used in professional hair salons or by private individuals, via the sale of kits.

For the purpose of the present disclosure, the expression “method for permanently reshaping” includes any long lasting method for shaping the hair, for curling, for relaxing, or for straightening.

As used herein, the term “keratin fibers” includes, for example, the hair, the eyelashes, and the eyebrows.

In the field of hair care, to obtain permanent reshaping of the hair, a chemical treating process is conventionally used and comprises, in a first step, opening the —S—S-disulphide bonds of the keratin (cystine) by utilizing a reducing composition comprising a reducing agent (i.e., a reduction step). Then, after having optionally rinsing the treated hair, reconstituting, in a second step, the disulphide bonds by applying, to the hair placed under tension beforehand (e.g., curlers or the like), an oxidizing composition (i.e., oxidation step, also referred to as fixing step) so as to give the hair, in the end, the desired shape. This technique allows, without distinction, to make the hair wavy, to relax it, or to straighten it. The new shape applied to the hair by the chemical treatment as described above is potentially long-lasting and able to withstand, for example, the action of washing with water or shampooing, as opposed to the simple conventional techniques of non-permanent reshaping, such as setting.

With respect to the first step of the permanent-wave process, the reducing compositions generally comprise, as reducing agents, sulphites, bisulphites or thiols. Among the latter, mention may be made of cysteine and its various derivatives, cysteamine and its derivatives, thiolactic acid, thioglycolic acid and its esters, for example, glyceryl monothioglycolate, and thioglycerol.

Regarding the oxidizing compositions required for carrying out the fixing step, compositions based on aqueous hydrogen peroxide or on alkali bromates are, in practice, commonly used.

One drawback of the permanent-wave techniques known to date is that repeated application thereof to the hair usually induces, in the long term, a gradual impairment of the quality of the hair, and for example, a gradual and marked impairment of the shine and of the cosmetic properties of the hair, such as the softness of the fibers, which have a tendency to become increasingly rough. In addition, in terms of the untangling of the fibers, the hair may become increasingly difficult to untangle. These drawbacks are also, for example, accentuated when the fixing step in the permanent reshaping process is carried out using a bromate.

In order to limit these changes to the hair, it is known to introduce conditioners directly into the reducing composition. For example, Japanese Patent Application Nos. H2-250814 and H9-151120 describe reducing compositions containing aminated silicones, that can optionally be in the form of a microemulsion.

The methods for permanently reshaping the hair that use such compositions, however, are not yet entirely satisfactory. For example, the quality and the liveliness of the curling are generally insufficient and short-lived, based in part on the conditioner directly combining with the reducing agent to impair the activity of the latter.

It would thus be desirable to have available compositions and methods for permanently reshaping keratin fibers, such as the hair, that reduce the mechanical and/or cosmetic degradation of the hair, while at the same time providing a satisfactory degree, quality and liveliness of curling.

The present inventors have discovered, surprisingly and unexpectedly, that using at least one elastomeric film-forming polymer as disclosed herein for protecting keratin fibers, during permanent shaping treatments or treatments for a relaxing operation, results in an improvement of the mechanical and cosmetic properties of the hair.

Another embodiment of the present disclosure relates to a method for permanently shaping or for relaxing keratin fibers comprising applying to the keratin fibers a composition comprising at least one elastomeric film-forming polymer as disclosed.

For example, the elastomeric film-forming polymer may be part of the reducing composition used during the shaping of permanent-waves.

Hair regularly subjected to permanent-wave processes is known to often lose its mechanical properties. This permanent-waved hair may be more brittle and may be difficult to shape; in addition, it may be difficult to obtain an even coloration on permanent-waved hair.

Cosmetic treatments aimed at relaxing keratin materials such as the hair, may also result in the mechanical properties of the keratin materials being impaired.

The use of the presently disclosed compositions comprising at least one elastomeric film-forming polymer during the shaping of permanent-waves or a hair-relaxing process make it possible to limit, or even eliminate, the degrading effects of permanent waves.

One aspect of the present disclosure is thus a cosmetic composition comprising, in a cosmetically acceptable medium, a combination

of at least one elastomeric film-forming polymer, wherein the film obtained by drying said at least one film-forming polymer at ambient temperature (22° C.±2° C.) and at a relative humidity of 55%±5%, has a mechanical profile comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%; and

of at least one compound chosen from keratin-reducing compounds and keratin-fixing compounds.

The present disclosure also relates to the use of this cosmetic composition for carrying out a permanent shaping process or a relaxing process; it may be a reducing composition or a fixing, such as oxidizing, composition.

The present disclosure also relates to a multicompartment kit comprising at least two compartments, wherein one of the compartments comprises a reducing composition comprising, in a cosmetically acceptable medium, a combination of at least one elastomeric film-forming polymer and at least one keratin-reducing compound, wherein the film obtained by drying said at least one film-forming polymer at ambient temperature and at a relative humidity of 55%±5%, has a mechanical profile comprising: (a) an elongation at break (ε_{b}) of greater than or equal to 800%, (b) an instantaneous recovery (Rᵢ) at least equal to 75%, after an elongation of 150%, and (c) a recovery (R₃₀₀) at 300 seconds of greater than 80%.

The present disclosure further relates to a multicompartment kit comprising at least two compartments, wherein one of the compartments comprises a fixing composition comprising, in a cosmetically acceptable medium, a combination of at least one elastomeric film-forming polymer and at least one keratin fixing compound, wherein the film obtained by drying said at least one film-forming polymer at ambient temperature and at a relative humidity of 55%±5%, has a mechanical profile comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

The present disclosure also relates to a method for permanently shaping or relaxing keratin fibers such as the hair, comprising applying to the fibers a cosmetic composition comprising, in a cosmetically acceptable medium, a combination of at least one elastomeric film-forming polymer and at least one compound chosen from keratin reducing compounds and keratin fixing compounds, wherein the film obtained by drying said at least one film-forming polymer at ambient temperature and at a relative humidity of 55%±5%, has a mechanical profile comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

The at least one elastomeric film-forming polymer may be used in combination with any of the conventional keratin-reducing compounds or with any of the conventional keratin-fixing compounds in any conventional cosmetic compositions for carrying out a permanent wave or a relaxing process.

For example, the at least one elastomeric film-forming polymer may be used for or in reducing compositions for carrying out permanent waves or for carrying out a relaxing process.

For the purpose of the present disclosure, the expression “film obtained by drying at ambient temperature (22° C.±2° C.) and at a relative humidity of 55%±5%” includes the film obtained, under these conditions, from a mixture containing 6% of active material (a.m.) of the at least one elastomeric film-forming polymer in a mixture of 30%, by weight of ethanol and 70%, by weight of water, relative to the total alcohol+water weight, the amount of mixture being adjusted so as to obtain, in a Teflon matrix, a film 500 μm±50 μm thick. The drying may be continued until the weight of the film no longer changes, which may represent approximately 12 days. The at least one film-forming polymer that are soluble or partially soluble in ethanol are tested in ethanol alone. The other polymers are tested in water alone, in a soluble or dispersed form.

For the purpose of the present disclosure, the elongation at break and the recovery rate are evaluated by the tests described below.

For carrying out the tensile test, the film is cut up into rectangle-shaped test pieces 80 mm long and 15 mm wide.

The tests are carried out on a device sold under the name Lloyd or sold under the name Zwick, under the same temperature and humidity conditions as for the drying, i.e. a temperature of 22° C.±2° C. and a relative humidity of 55%±5%.

The test pieces are drawn at a speed of 20 mm/min and the distance between the jaws is 50±1 mm.

To determine the instantaneous recovery (Rᵢ), the following procedure was carried out:

the test piece is drawn by 150% (εₘₐₓ), i.e. 1.5 times its initial length (l₀),

the stress is released by applying a return speed equal to the tensile speed, i.e. 20 mm/min, and the percentage elongation of the test piece is measured, after returning to zero constraint (εᵢ).

The % instantaneous recovery (Rᵢ) is given by the formula below:
Rᵢ=((εₘₐₓ−εᵢ)/εₘₐₓ)×100

To determine the recovery at 300 seconds, the test piece, having undergone the above operations, is maintained at zero constraint for a further 300 seconds, and its percentage elongation (ε₃₀₀ₛ) is measured.

The % recovery at 300 seconds (R₃₀₀ₛ) is given by the formula below:
R₃₀₀ₛ=((εₘₐₓ−ε₃₀₀ₛ)/εₘₐₓ)×100

For example, the at least one film-forming polymer of the composition according to the disclosure, may be optionally combined with a plasticizer and/or a film-forming agent, such that it forms, under the conditions of the above test, a film having an elongation at break ranging from 800% to 3000%; an instantaneous recovery of 75% to 100%; and a recovery at 300 seconds ranging from 85% to 100%.

In the compositions in accordance with the disclosure, the at least one elastomeric film-forming polymer or the blend of elastomeric film-forming polymers may, for example, be present in a concentration ranging from 0.05% to 20% by weight, such as from 0.1% to 15% by weight, and for example from 0.25% to 10% by weight, relative to the total weight of the composition.

Further for example, the at least one elastomeric film-forming polymer is chosen from polyurethanes, polyvinyl alcohols, polymers comprising at least one (meth)acrylic unit, and combinations thereof. It may be in the form of a homopolymer or of a copolymer. The at least one film-forming polymer may be in a non-crosslinked form in the composition.

The composition may, if necessary, further comprise a plasticizer and/or an additional agent for facilitating the formation of a film of the at least one elastomeric film-forming polymer on keratin materials, the function of which is to modify the properties of the elastomeric polymer(s). Such an additional film-forming agent may be chosen from any of the compounds known to those skilled in the art as being capable of performing the desired function, and may, for example, be chosen from plasticizers and coalescence agents. The at least one elastomeric film-forming polymer, optionally combined with a plasticizer and/or an agent for facilitating film formation, is capable of forming a film, after evaporation of the cosmetic medium. This evaporation may be carried out in the open air or by supplying heat such as using a dryer.

Non-limiting examples of plasticizers and/or additional agents for facilitating film formation on keratin materials, may be made of those described in document FR-A-2 782 917. For example, this plasticizer and/or additional agent is chosen from the usual plasticizers or coalescence agents, such as:

glycols and their derivatives, such as diethylene glycol ethyl ether, diethylene glycol methyl ether, diethylene glycol butyl ether or else diethylene glycol hexyl ether ethylene glycol ethyl ether, ethylene glycol butyl ether, ethylene glycol hexyl ether or pentylene glycol,

glyceryl esters,

derivatives of propylene glycol, and for example, propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,

acid esters, for example, carboxylic acid esters, such as citrates, phthalates, adipates, carbonates, tartrates, phosphates or sebacates, and

mixtures thereof.

The amount of plasticizer and/or additional film-forming agent may be chosen by those skilled in the art based on their general knowledge, so as to obtain a polymeric system (at least one elastomeric polymer+plasticizer and/or additional film-forming agent) that produces a film having the desired mechanical properties, while at the same time allowing the composition to conserve the desired cosmetic properties. In practice, the amount of plasticizer and/or additional film-forming agent ranges from 0.01% to 25%, by weight of the total weight of the composition, and for example, from 0.01% to 15%.

The reducing compositions for carrying out permanent waves may comprise any composition already known in itself as a reducing composition.

For example, the reducing compositions that may be used for carrying out permanent waves comprise, as keratin-reducing agents, sulphites and/or bisulphites, such as alkali metal, alkaline earth metal or ammonium sulphites and/or bisulphites, or for example, thiols. Among the thiols, those most commonly used are cysteine and its various derivatives (e.g., N-acetylcysteine), cysteamine and its various derivatives (e.g., C₁-C₄ acylated derivatives such as N-acetylcysteamine or N-propionylcysteamine), thiolactic acid and its esters (e.g., glyceryl monothiolactate), thioglycolic acid and its esters, such as glyceryl monothioglycolate or glycol monothioglycolate, and thioglycerol. Mention may also be made, for example, of the following reducing agents: N-mercaptoalkylamides of sugars such as N-(2-mercaptoethyl)gluconamide, β-mercaptopropionic acid and its derivatives, thiomalic acid, pantetheine, the N-(mercaptoalkyl)-ω-hydroxyalkylamides described in European Patent Application No. EP-A-354 835 and the N-mono- or N,N-dialkylmercapto-4-butyramides described in European Patent Application No. EP-A-368 763, the aminomercaptoalkylamides described in European Patent Application No. EP-A-432 000 and the alkylaminomercaptoalkylamides described in European Patent Application EP-A-514 282, and the mixture of 2-hydroxypropyl thioglycolate (2/3) and 2-hydroxy-1-methylethyl thioglycolate (67/33) described in French Patent Application No. FR-A-2 679 448.

These keratin-reducing agents are generally used in cosmetically acceptable compositions, which are, moreover, already known in the art relating to curling formulations intended to carry out the first step (i.e., the reduction step) of a permanent-waving process. Thus, normal and conventional additives, that may be used alone or as mixtures, mention may be made, for example, of surfactants of non-ionic, anionic, cationic or amphoteric type, and among these, mention may be made, for example, of alkyl sulphates, alkylbenzene sulphates, alkyl ether sulphates, alkylsulphonates, quaternary ammonium salts, alkylbetaines, oxyethylenated alkylphenols, fatty acid alkanolamides, oxyethylenated fatty acid esters, and other non-ionic surfactants of the hydroxypropyl ether type.

When the reducing composition comprises at least one surfactant, the at least one surfactant is present at a maximum concentration of 30% by weight, and for example, ranging from 0.5% and 10% by weight, relative to the total weight of the reducing composition.

With the aim of improving the cosmetic properties of the hair, or alternatively of reducing or preventing degradation thereof, the reducing composition may also, in addition to the polyguanidine-derived compound, comprise at least one treating agent of cationic, anionic, non-ionic or amophoteric nature.

Among the at least one treating agent, mention may be made, for example, of those described in French Patent Application Nos. 2 598 613 and 2 470 596. Treating agents that may also be used include volatile or non-volatile, linear or cyclic silicones and mixtures thereof, polydimethylsiloxanes, quaternized polyorganosiloxanes such as those described in French Patent Application No. 2 535 730, polyorganosiloxanes comprising aminoalkyl groups modified with alkoxycarbonylalkyl groups such as those described in U.S. Pat. No. 4,749,732, polyorganosiloxanes such as the copolymer of polydimethylsiloxane-polyoxyalkyl of the dimethicone copolyol type, a polydimethylsiloxane comprising stearoxy-(stearoxy dimethicone) end groups, a polydimethylsiloxane-dialkylammonium acetate copolymer or a polydimethylsiloxane-polyalkylbetaine copolymer described in British Patent Application No. 2,197,352, polyorganosiloxanes modified with mercapto or mercaptoalkyl groups such as those described in French Patent No. 1,530,369 and in European Patent Application No. 295 780, and also silanes such as stearoxytrimethylsilane.

The reducing composition may further comprise other additional treating ingredients such as cationic polymers, for example, those used in the compositions of French Patent Nos. 79 32078 (FR-A-2 472 381) and 80 26421 (FR-A-2 495 931), or else cationic polymers of the ionene type, such as those used in the compositions of Luxembourg Patent No. 83703, basic amino acids (such as, lysine or arginine) or acidic amino acids (such as, glutamic acid or aspartic acid), peptides and their derivatives, protein hydrolysates, waxes, swelling agents, penetrating agents or agents for reinforcing the effectiveness of the reducing agent, such as the mixture SiO₂/PDMS (polydimethylsiloxane), dimethylisosorbitol, urea and its derivatives, pyrrolidone, N-alkylpyrrolidones, thiamorpholinone, alkylene glycol alkyl ethers or dialkylene glycol alkyl ethers, for example, propylene glycol monomethyl ether, dipropylene glycol monomethyl ether, ethylene glycol monoethyl ether and diethylene glycol monoethyl ether, C₃-C₆ alkanediols such as, for example, 1,2-propanediol and 1,2-butanediol, 2-imidazolidinone, and other compounds such as fatty alcohols, lanolin derivatives, active ingredients such as pantothenic acid, anti-hair loss agents, anti-dandruff agents, thickeners, suspending agents, sequestering agents, opacifiers, dyes, sunscreens, fragrances, and preserving agents.

In permanent-wave reducing compositions, the reducing agents as mentioned above may be present at a concentration ranging from 1% to 30% by weight, and for example, from 5% to 20% by weight, relative to the total weight of the reducing composition.

The reducing composition may be in a form chosen from a thickened and non-thickened lotion, a cream, a gel, and any other appropriate form.

The reducing composition may also be of the exothermic type, i.e. causing a certain heating when applied to the hair, which may be pleasant for the individual undergoing the permanent wave or the relaxing process.

The reducing composition may further comprise a solvent chosen from, for example, ethanol, propanol and isopropanol, and alternatively glycerol, at a maximum concentration of 20% by weight, relative to the total weight of the composition.

The vehicle, i.e., cosmetic medium, for the presently disclosed compositions is, for example, water or an aqueous-alcoholic solution of a lower alcohol such as ethanol, isopropanol or butanol.

When the compositions are intended for a hair relaxing or straightening process, the reducing composition is, for example, in the form of a thick cream so as to keep the hair as straight as possible. These creams are prepared in the form of “heavy” emulsions.

For example, in order to obtain a cream, an aqueous phase comprising the polyguanidine-derived compound and, optionally, other ingredients or adjuvants, in solution, and an oily phase may be emulsified.

The oily phase may comprise various products, such as paraffin oil, liquid petroleum jelly, sweet almond oil, avocado oil, olive oil, fatty acid esters such as glyceryl monostearate, ethyl palmitate or isopropyl palmitate, or alkyl myristates such as propyl, butyl or cetyl myristates. Fatty alcohols such as cetyl alcohol or waxes such as, for example, beeswax may also be added.

The reducing compositions may also be made of liquids or gels comprising thickeners such as carboxyvinyl polymers or copolymers that “bond” the hair and keep it in the smoothed out position during the period of application.

Finally, the reducing compositions may further comprise at least one disulphide known for its use in a reducing composition for self-neutralizing permanent-waving.

Among such known disulphides, mention may be made, for example, of dithioglycolic acid, dithioglycerol, cystamine, N,N′-diacetylcystamine, cystine, pantethine, and disulphides of the N-(mercaptoalkyl)-ω-hydroxyalkylamides described in European Patent Application No. EP 354 835, disulphides of the N-mono- or N,N-dialkylmercapto-4-butyramides described in European Patent Application No. EP 368 763, disulphides of the aminomercaptoalkylamides described in European Patent Application No. EP 432 000, and disulphides of the alkylaminomercaptoalkylamides described in European Patent Application No. EP 514 282. These disulphides are generally present in a molar ratio from 0.5 to 2.5, and for example, from 1 to 2, relative to the reducing agent (e.g., U.S. Pat. No. 3,768,490).

The pH values of the reducing compositions may be adjusted conventionally by the addition either of basifying agents, for example, aqueous ammonia, monoethanolamine, diethanolamine, triethanolamine, isopropanolamine, 1,3-propanediamine; an alkali metal carbonate or bicarbonate or an ammonium carbonate or bicarbonate; an organic carbonate such as guanidine carbonate (carbonated reducing compositions); and else an alkali metal hydroxide; or of acidifying agents, for example, hydrochloric acid, acetic acid, lactic acid or boric acid. It being possible for all these compounds to be taken alone or as a mixture.

The compositions comprising at least one elastomeric film-forming polymer and at least one reducing compound chosen from the sulphites or the bisulphites as defined above, may be applied repeatedly to the hair so as to carry out permanent waves or relaxing processes, without a considerable modification of the behavior of this hair being observed, for example, the hair's ability to subsequently be correctly dyed.

It may generally be observed that, on hair that has undergone a few permanent-wave or relaxing processes (e.g., three at most), the coloration will be much more pronounced than that obtained on the same hair, that has not undergone a permanent wave. This, therefore, may present variations between the hair that was originally subjected to a permanent wave and the hair that has regrown (i.e., poor combination between the original permanent-waved hair and the non-permanent-waved hair that has regrown).

It is also observed that dyeing becomes difficult, or even almost impossible, if the hair to be dyed has previously undergone many permanent-wave or relaxing processes, for example, more than five permanent-waves.

The cosmetic compositions used in the context of the present disclosure may be both ready-to-use compositions and concentrates that have to be diluted before use. The cosmetic compositions are not, therefore, limited to a concentration range of the at least one film-forming polymer.

Generally, in the cosmetic composition used, the concentration of the at least one elastomeric film-forming polymer ranges from 0.001% to 25% by weight, and for example, from 0.1% to 10% by weight, relative to the total weight of the composition.

These compositions may be packaged in various forms, such as in bottles optionally equipped with a pump, heating bags, tubes or simple pots for scooping the composition with the hands. The compositions in accordance with the disclosure may be in the form chosen from a cream, a gel, a water-in-oil and oil-in-water emulsion, a lotion, a spray, a mousse, a wax, and mixtures thereof.

In at least one embodiment, the pH of the aqueous phase ranges from 2 to 11, such as from 3 to 10, and for example, from 5 to 8.

The composition of the present disclosure may have a dynamic viscosity, measured at ambient temperature and atmospheric pressure, of less than 200 cps, and for example, from 180 cps to 10 cps. This viscosity may be measured by any known method, and for example, using a Rheomat 180, such as at 25° C. and with a shear rate of 1 s⁻¹.

Of course, those skilled in the art will take care to choose the optional compound(s) to be added to the composition according to the disclosure in such a way that the advantageous properties intrinsically associated with the composition in accordance with the disclosure are not, or are not substantially, impaired by the envisaged addition.

The compositions in accordance with the disclosure may be applied to keratin materials such as the hair, skin, and eyelashes, in the dry or wet state.

Other than in the examples, or where otherwise indicated, all numbers expressing quantities of ingredients, reaction conditions, and so forth used in the specification and claims are to be understood as being modified in all instance by the term “about.” Accordingly, unless indicated to the contrary, the numerical parameters set forth in the following specification and attached claims are approximations that may vary depending upon desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contain certain errors necessarily resulting from the standard deviation found in their respective testing measurements. The following example is intended to illustrate the present disclosure without limiting the scope as a result.

The amounts are given as percentage by mass, and A.M. signifies active material.

Example of a permanent-wave composition:

| | |
|---|---|
| Polyurethane (NMDEA¹⁾/PTMO 2900²⁾/IPDI³⁾ - 3/1/4) | 4% AM |
| Thioglycolic acid | 9 g |
| Aqueous ammonia pH adjusted to 8.4 | pH 8.4 |
| Water | qs 100% AM |

¹⁾N-methyldiethanolamine
²⁾Poly(tetramethylene oxide) having a weight-average mass of 2900
³⁾Isophorone diisocyanate

What is claimed is:

1. A cosmetic composition comprising, in a cosmetically acceptable medium,
at least one elastomeric film-forming polymer, wherein the film obtained by drying said at least one film-forming polymer at ambient temperature and at a relative humidity of 55%±5%, has a mechanical profile comprising: (a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%; and
at least one compound chosen from keratin-reducing compounds and keratin-fixing compounds.

2. The composition according to claim 1, wherein the at least one film-forming polymer is soluble in a medium chosen from an aqueous and aqueous-alcoholic medium.

3. The composition according to claim 1, wherein at a relative humidity from 30% to 80%, the elongation at break of the film obtained ranges from 400% to 1200% and/or the film obtained has an instantaneous recovery ranging from 57% to 93%.

4. The composition according to claim 3, wherein the composition has a dynamic viscosity, measured at ambient temperature and at atmospheric pressure, of less than 200 cps.

5. The composition according to claim 1, wherein the at least one film-forming polymer is chosen from polyurethanes, polyvinyl alcohols, polymers comprising at least one (meth)acrylic unit, and mixtures thereof.

6. The composition according to claim 5, wherein the at least one compound is chosen from keratin-reducing compounds.

7. The composition according to claim 6, wherein the keratin-reducing compounds are chosen from thiols, cysteamine and derivatives thereof, thiolactic acid and esters thereof, thioglycolic acid and esters thereof, and mixtures thereof.

8. The composition according to claim 7, wherein the thiols comprise cysteine and derivatives thereof.

9. The composition according to claim 1, wherein the at least one film-forming polymer is present in the composition in an amount ranging from 0.001% to 25% by weight, relative to the total weight of the cosmetic composition.

10. The composition according to claim 9, wherein the at least one film-forming polymer is present in the composition in an amount ranging from 0.1% to 10% by weight, relative to the total weight of the cosmetic composition.

11. The composition according to claim 1, wherein the cosmetic composition further comprises at least one agent chosen from non-ionic, anionic, cationic and amphoteric surfactants, and non-ionic, anionic, cationic and amphoteric treating agents.

12. A method for permanently shaping or for relaxing keratin fibers comprising applying to the keratin fibers a cosmetic composition comprising, in a cosmetically acceptable medium, at least one elastomeric film-forming polymer, wherein the film obtained by drying said at least one film-forming polymer at ambient temperature and at a relative humidity of 55%±5%, has a mechanical profile comprising:
(a) an elongation at break (sb) of greater than or equal to 800%,
(b) an instantaneous recovery (Rᵢ) at least equal to 75%, after an elongation of 150%, and
(c) a recovery (R₃₀₀) at 300 seconds of greater than 80%; and
at least one compound chosen from keratin-reducing compounds and keratin-fixing compounds.

13. The method according to claim 12, wherein the cosmetic composition is a reducing composition.

14. The method according to claim 12, wherein the cosmetic composition is a fixing composition for permanently shape keratin fibers.

15. The method according to claim 12, wherein the at least one film-forming polymer is present in the composition in an amount ranging from 0.001% to 25% by weight, relative to the total weight of the cosmetic composition.

16. The method according to claim 15, wherein the at least one film-forming polymer is present in the composition in an amount ranging from 0.1% to 10% by weight, relative to the total weight of the cosmetic composition.

17. The method according to claim 12, wherein the cosmetic composition further comprises at least one agent chosen from non-ionic, anionic, cationic and amphoteric surfactants, and non-ionic, anionic, cationic and amphoteric treating agents.

18. A kit comprising at least two compartments, wherein one of the compartments comprises a reducing cosmetic composition comprising, in a cosmetically acceptable medium, at least one elastomeric film-forming polymer, wherein the film obtained by drying said at least one film-forming polymer at ambient temperature and at a relative humidity of 55%±5%, has a mechanical profile comprising:
(a) an elongation at break (sb) of greater than or equal to 800%,
(b) an instantaneous recovery (Rᵢ) at least equal to 75%, after an elongation of 150%, and
(c) a recovery (R₃₀₀) at 300 seconds of greater than 80%; and
at least one keratin-reducing compound.

19. A kit comprising at least two compartments, wherein one of the compartments comprises a fixing cosmetic composition comprising, in a cosmetically acceptable medium, at least one elastomeric film-forming polymer, wherein the film obtained by drying said at least one film-forming polymer at ambient temperature and at a relative humidity of 55%±5%, has a mechanical profile comprising::
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%; and
at least one keratin-fixing compound.
